(19) United States
(12) Patent Application Publication
(10) Pub. No.: US 2018/0301637 A1
(43) Pub. Date: Oct. 18, 2018

(54) ORGANIC ELECTROLUMINESCENT COMPONENT

(71) Applicants: GUANGDONG AGLAIA OPTOELECTRONIC MATERIALS CO., LTD, Foshan, Guangdong (CN); BEIJING AGLAIA TECHNOLOGY DEVELOPMENT CO., LTD., Beijing (CN)

(72) Inventors: Zhe LI, Foshan (CN); Kam-Hung LOW, Foshan (CN); Lei DAI, Beijing (CN); Lifei CAI, Beijing (CN)

(73) Assignees: GUANGDONG AGLAIA OPTOELECTRONIC MATERIALS CO., LTD ., Foshan, Guangdong (CN); BEIJING AGLAIA TECHNOLOGY DEVELOPMENT CO., LTD., Beijing (CN)

(21) Appl. No.: 15/735,173

(22) PCT Filed: Apr. 27, 2016
(86) PCT No.: PCT/CN2016/080319

§ 371 Date: Dec. 9, 2017

(30) Foreign Application Priority Data
Jun. 10, 2015 (CN) 2015103175159
Mar. 22, 2016 (CN) 2016101690906

(51) Int. Cl. H01L 51/00; C07D 209/86; C09K 11/06

(52) U.S. Cl. CPC H01L 51/0072; H01L 51/5088; C09K 11/06; C07D 209/86

(57) ABSTRACT

The present invention relates to an OLED, comprising an anode, a cathode and one or more organic layers, and at least one of the organic layers comprising a material having the following chemical formula I or II. Since the compound having the formula (I) in the present invention has electron-withdrawing groups and electron-donating groups with high stability, stable chemical bonds exist between them, without strong conjugation. With these characteristics, the compound having the formula (I) has higher fluorescence quantum efficiency and excellent charge transport ability, and its emission spectra are closer to the international standard of dark blue, to facilitate to the realization of full-color displays with higher color purity.

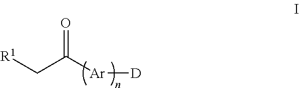

### TECHNICAL FIELD

The present invention relates to an organic light emitting device (OLED), belonging to the field of organic optoelectronics.

### BACKGROUND ART

OLED, as a new type of display technology, has unique advantages such as self-illumination, wide viewing angle, low power consumption, high efficiency, thin, rich colors, fast response, used to make flexible and transparent light-emitting device, etc., therefore, the OLED technology can be applied to novel flat panel displays, face light source lighting and wearable equipments, etc., or can be used as a backlight of LCD.

After years of development, OLEDs technology (OLED) has reached the level of marketization. However, the widely used high-efficiency phosphorescent materials need to use iridium, platinum and other rare precious metals, which is one of the important factors of high costs of OLEDs. In order to reduce the cost of OLED materials, it is possible to develop high-cost and high-stability materials that can make full use of the triplet energy levels of the electrical excitation devices, such as compounds with thermal excitation delayed fluorescence, which is one of the promising methods to solve the high-cost problems of high-performance OLED materials.

### SUMMARY OF THE INVENTION

The object of the present invention is to provide an OLED made of new material. This kind of materials can be used to make dark blue OLEDs closer to the international standard, to achieve the full-color displays with higher color purity.

The OLED described in the invention comprises an anode, a cathode and one or more organic layers, and at least one of the organic layers comprising a material having the following chemical formula (I):

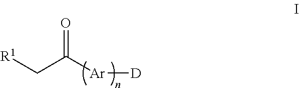

Wherein, Ar is unsubstituted or at least one R4-substituted benzene ring, naphthalene ring, anthracene ring, n=0-3;

D is an electron donating group containing a nitrogen atom and is one of the following groups:

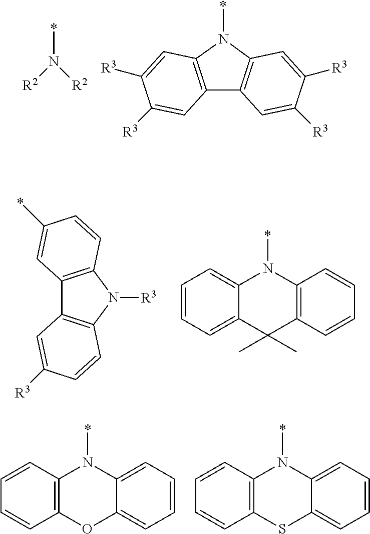

When D is —N(R²)₂, n is not zero;

R¹, R³, R⁴ are each independently selected from hydrogen H, deuterium D, fluorine F, chlorine Cl, bromine Br, iodine I, hydroxy OH, cyano CN, amino NH2, nitro NO₂, cyclic or non-cyclic alkyl group containing 1 to 20 carbon atoms, cyclic or non-cyclic alkoxy containing 1 to 20 carbon atoms, C6-C40 R⁵-substituted or unsubstituted aryl containing one or more substituents, C6-C40 R⁵-substituted or unsubstituted aryl alkyl containing one or more substituents, C5-C40 R⁵-substituted or unsubstituted heteroaryl groups containing one or more heteroatoms; two or more of the R²-R⁴ groups may be bonded to each other to form a ring;

R² is independently selected from C6-C40 R⁵ substituted or unsubstituted aryl containing one or more substituents, C6-C40 R⁵ substituted or unsubstituted aryl alkyl containing one or more substituents, C5-C40 R⁵-substituted or unsubstituted heteroaryl groups containing one or more heteroatoms;

R⁵ is selected from fluorine F, chlorine Cl, bromide Br, iodine I, hydroxy OH, cyano CN, amino NH₂, nitro NO₂, cyclic or non-cyclic alkyl containing 1 to 20 carbon atoms;

The heteroatoms are B, O, S, Se, N, P.

Preferably, R¹, R³, R⁴ are each independently selected from hydrogen H, deuterium D, fluorine F, chlorine Cl, bromine Br, iodine I, hydroxy OH, cyano CN, amino NH2, nitro NO₂, alkyl group containing 1 to 4 carbon atoms, alkoxy containing 1 to 4 carbon atoms, C6-C25 R⁵-substituted or unsubstituted aryl containing one or more substituents, C6-C25 R⁵-substituted or unsubstituted aryl alkyl containing one or more substituents, C5-C25 R⁵-substituted or unsubstituted heteroaryl groups containing one or more heteroatoms; R² is independently selected from C6-C25 R⁵ substituted or unsubstituted aryl containing one or more substituents, C6-C25 R⁵ substituted or unsubstituted aryl alkyl containing one or more substituents, C5-C25 R⁵-substituted or unsubstituted heteroaryl groups containing one or more heteroatoms; two or more of the R¹-R⁴ groups may be bonded to each other to form a ring;

R⁵ is selected from hydrogen H, fluorine F, chlorine Cl, bromide Br, iodine I, hydroxy OH, cyano CN, amino NH₂, nitro NO₂, alkyl containing 1 to 4 carbon atoms; and the heteroatoms are O, S, N.

Ar is unsubstituted or one R4 substituted benzene ring, naphthalene ring, anthracene ring.

More preferably, R¹, R³, R⁴ are each independently selected from hydrogen H, fluorine F, chlorine Cl, bromine Br, alkyl group containing 1 to 4 carbon atoms, C6-C25 R⁵-substituted or unsubstituted aryl containing one or more substituents, C6-C25 R⁵-substituted or unsubstituted aryl alkyl containing one or more substituents, C5-C25 R⁵-substituted or unsubstituted heteroaryl groups containing one or more heteroatoms; R² is independently selected from C6-C25 R⁵ substituted or unsubstituted aryl containing one or more substituents, C5-C25 R⁵-substituted or unsubstituted heteroaryl groups containing one or more heteroatoms; two or more of the R¹-R⁴ groups may be bonded to each other to form a ring;

R⁵ is selected from hydrogen H, fluorine F, chlorine Cl, bromide Br, alkyl containing 1 to 4 carbon atoms;

The heteroatoms are O, S, N.

Further preferably,

R¹ is selected from alkyl containing 1 to 4 carbon atoms, R5 substituted or unsubstituted phenyl, naphthyl, anthryl containing one or more substituents;

R² is independently R5 substituted or unsubstituted phenyl or naphthyl or anthryl containing one or more substituents;

R³ is selected from hydrogen H, fluorine F, chlorine Cl, bromine Br, iodine I, alkyl having 1 to 4 carbon atoms, R5 substituted or unsubstituted phenyl or naphthyl or anthryl containing one or more substituents;

R⁴ is selected from hydrogen H, fluorine F, chlorine Cl, bromine Br, iodine I, alkyl, phenyl, naphthyl containing 1 to 4 carbon atoms;

R⁵ is selected from hydrogen H, alkyl containing 1 to 4 carbon atoms.

In particular, preferably,

R¹ is selected from alkyl containing 1 to 4 carbon atoms;

R² is independently phenyl or naphthyl or anthryl;

R³ is selected from hydrogen H, phenyl or naphthyl or anthryl containing 1 to 4 carbon atoms;

R⁴ is selected from hydrogen H, phenyl or naphthyl or anthryl containing 1 to 4 carbon atoms.

The material having the formula I can be selected from one of the following structures:

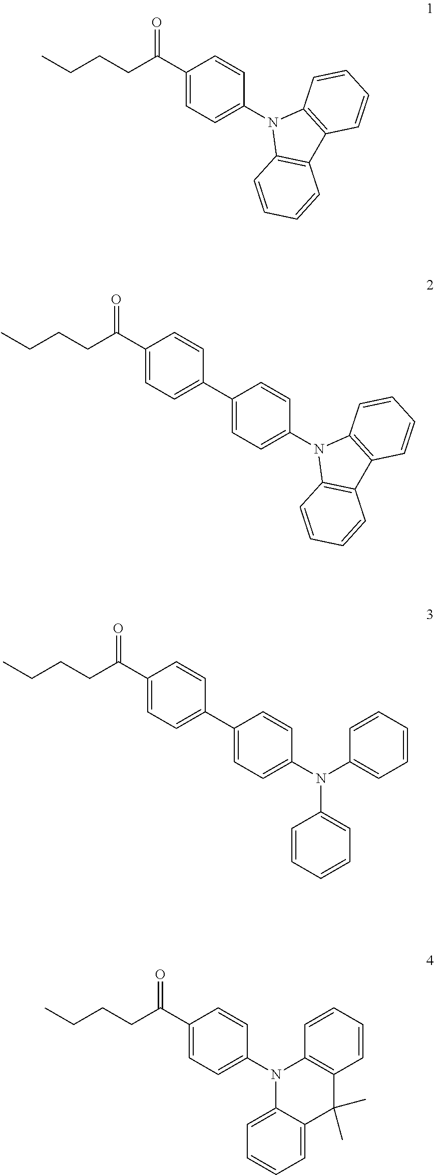 [SMILES: CCCCC(=O)c1ccc(-n2c3ccccc3c3ccccc32)cc1 ; CCCCC(=O)c1ccc(-c2ccc(-n3c4ccccc4c4ccccc43)cc2)cc1 ; CCCCC(=O)c1ccc(-c2ccc(N(c3ccccc3)c3ccccc3)cc2)cc1 ; CCCCC(=O)c1ccc(N2c3ccccc3C(C)(C)c3ccccc32)cc1]

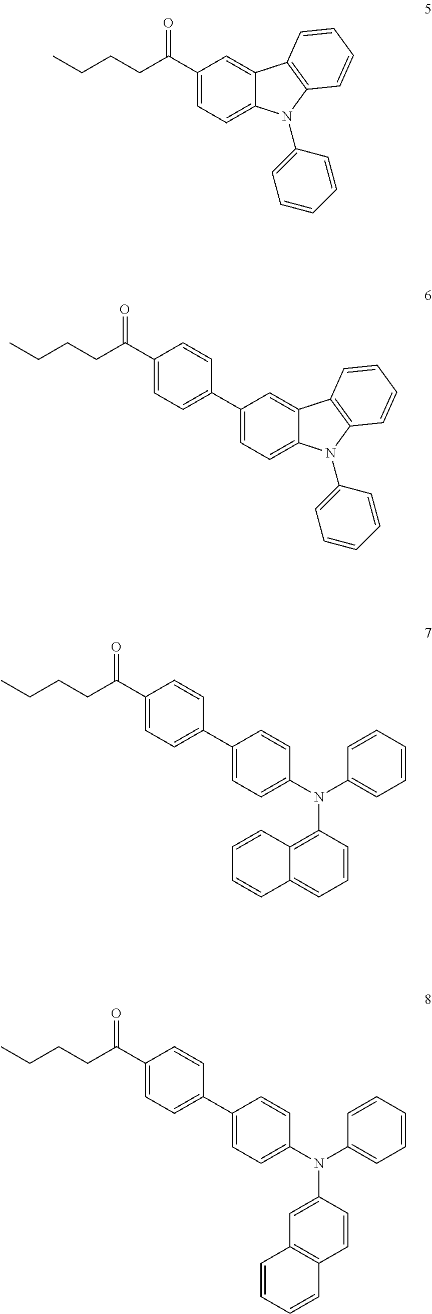 [SMILES: CCCCC(=O)c1ccc2c(c1)c1ccccc1n2-c1ccccc1 ; CCCCC(=O)c1ccc(-c2ccc3c(c2)c2ccccc2n3-c2ccccc2)cc1 ; CCCCC(=O)c1ccc(-c2ccc(N(c3ccccc3)c3cccc4ccccc34)cc2)cc1 ; CCCCC(=O)c1ccc(-c2ccc(N(c3ccccc3)c3ccc4ccccc4c3)cc2)cc1]

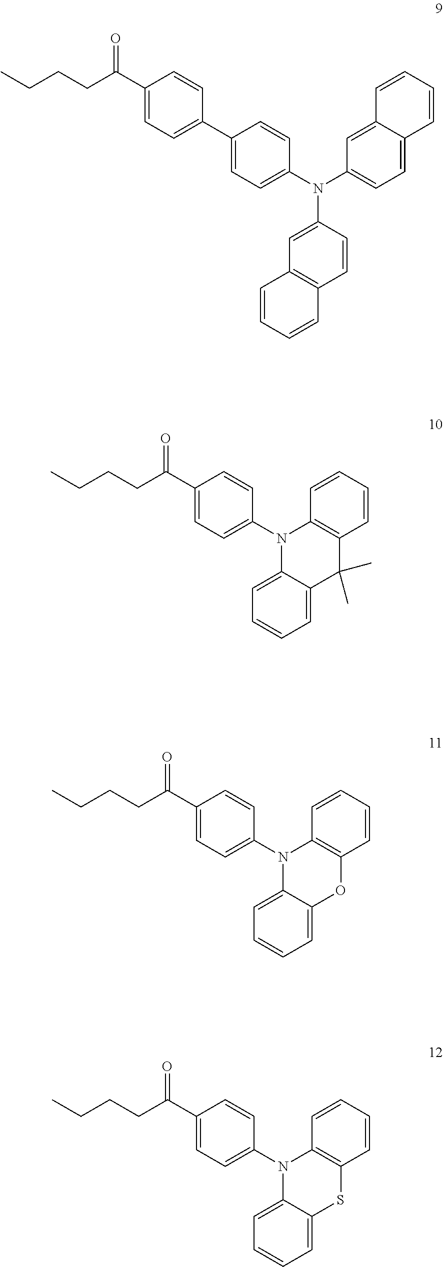

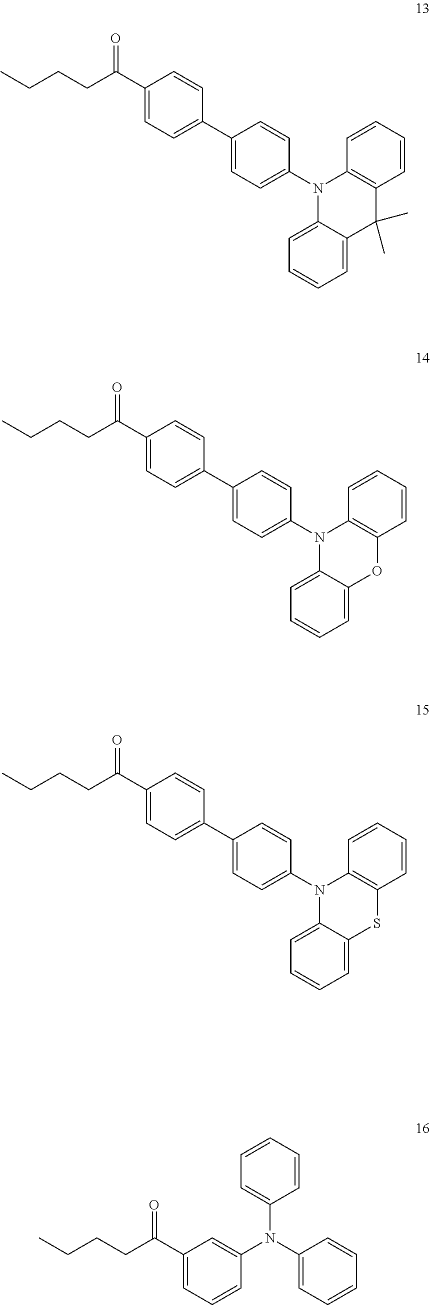

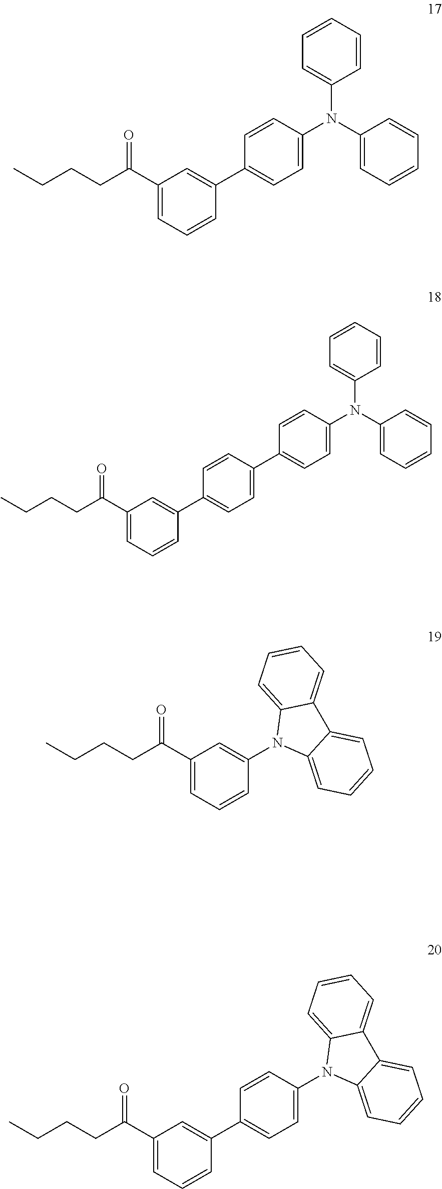 [SMILES: CCCCC(=O)c1cccc(-c2ccc(N(c3ccccc3)c3ccccc3)cc2)c1 ; CCCCC(=O)c1cccc(-c2ccc(-c3ccc(N(c4ccccc4)c4ccccc4)cc3)cc2)c1 ; CCCCC(=O)c1cccc(-n2c3ccccc3c3ccccc32)c1 ; CCCCC(=O)c1cccc(-c2ccc(-n3c4ccccc4c4ccccc43)cc2)c1]

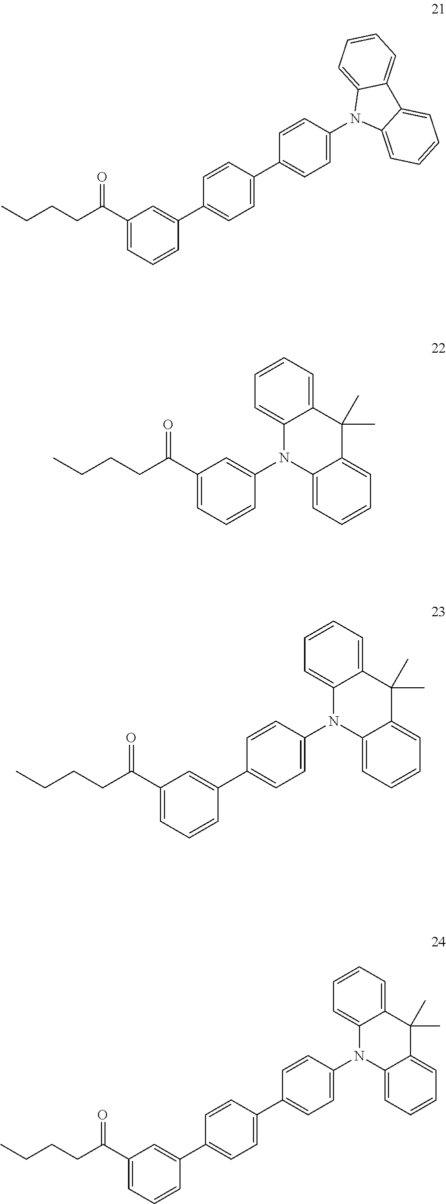

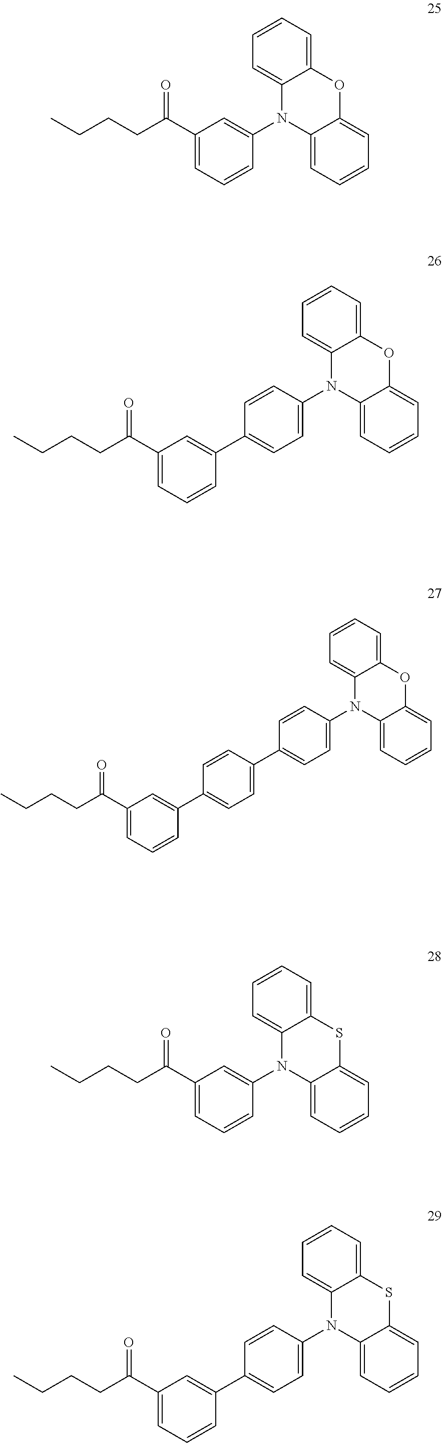 [SMILES: CCCCC(=O)c1cccc(N2c3ccccc3Oc3ccccc32)c1 ; CCCCC(=O)c1cccc(-c2ccc(N3c4ccccc4Oc4ccccc43)cc2)c1 ; CCCCC(=O)c1cccc(-c2ccc(-c3ccc(N4c5ccccc5Oc5ccccc54)cc3)cc2)c1 ; CCCCC(=O)c1cccc(N2c3ccccc3Sc3ccccc32)c1 ; CCCCC(=O)c1cccc(-c2ccc(N3c4ccccc4Sc4ccccc43)cc2)c1]

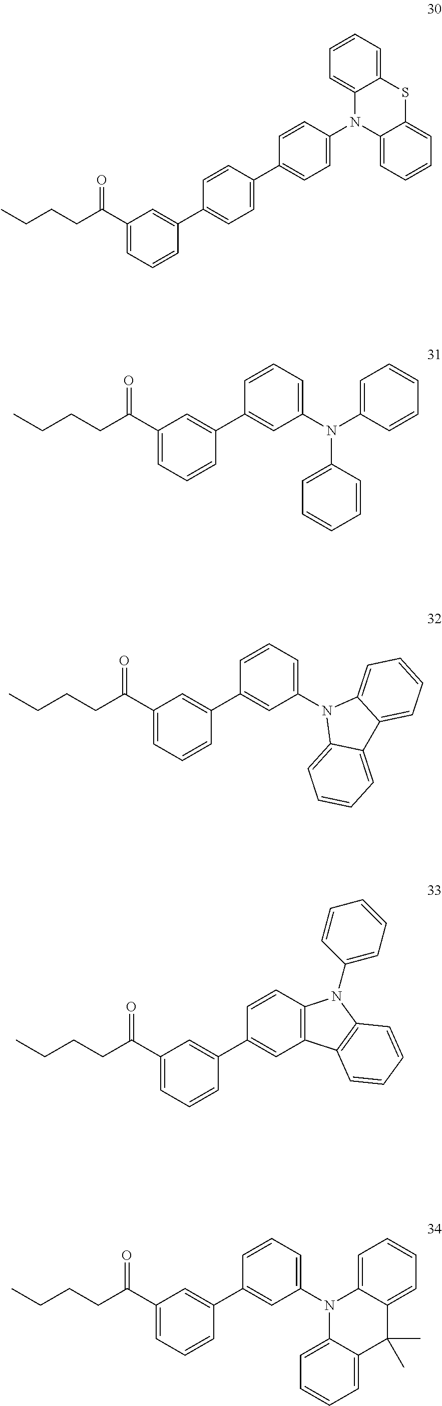 [SMILES: CCCCC(=O)c1cccc(-c2ccc(-c3ccc(N4c5ccccc5Sc5ccccc54)cc3)cc2)c1 ; CCCCC(=O)c1cccc(-c2cccc(N(c3ccccc3)c3ccccc3)c2)c1 ; CCCCC(=O)c1cccc(-c2cccc(-n3c4ccccc4c4ccccc43)c2)c1 ; CCCCC(=O)c1cccc(-c2ccc3c(c2)c2ccccc2n3-c2ccccc2)c1 ; CCCCC(=O)c1cccc(-c2cccc(N3c4ccccc4C(C)(C)c4ccccc43)c2)c1]

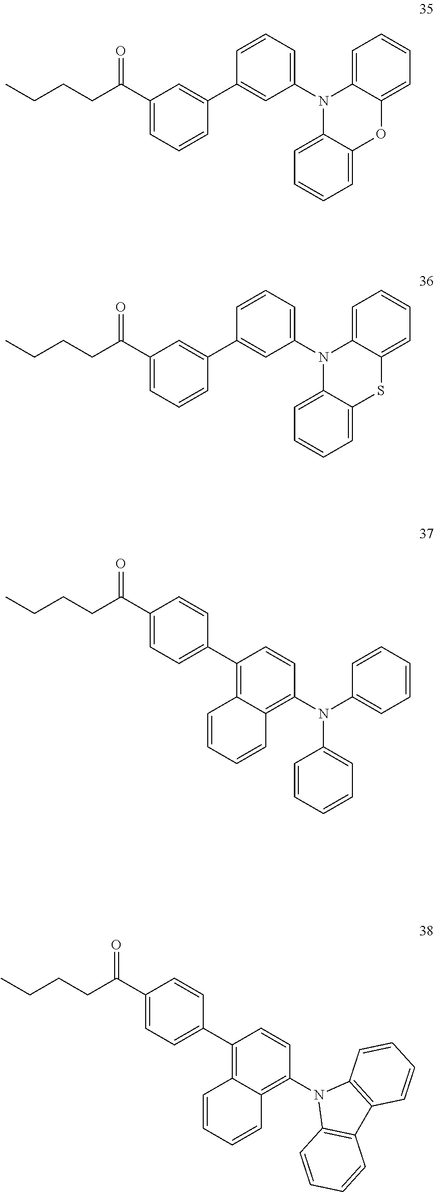 [SMILES: CCCCC(=O)c1cccc(-c2cccc(N3c4ccccc4Oc4ccccc43)c2)c1 ; CCCCC(=O)c1cccc(-c2cccc(N3c4ccccc4Sc4ccccc43)c2)c1 ; CCCCC(=O)c1ccc(-c2ccc(N(c3ccccc3)c3ccccc3)c3ccccc23)cc1 ; CCCCC(=O)c1ccc(-c2ccc(-n3c4ccccc4c4ccccc43)c3ccccc23)cc1]

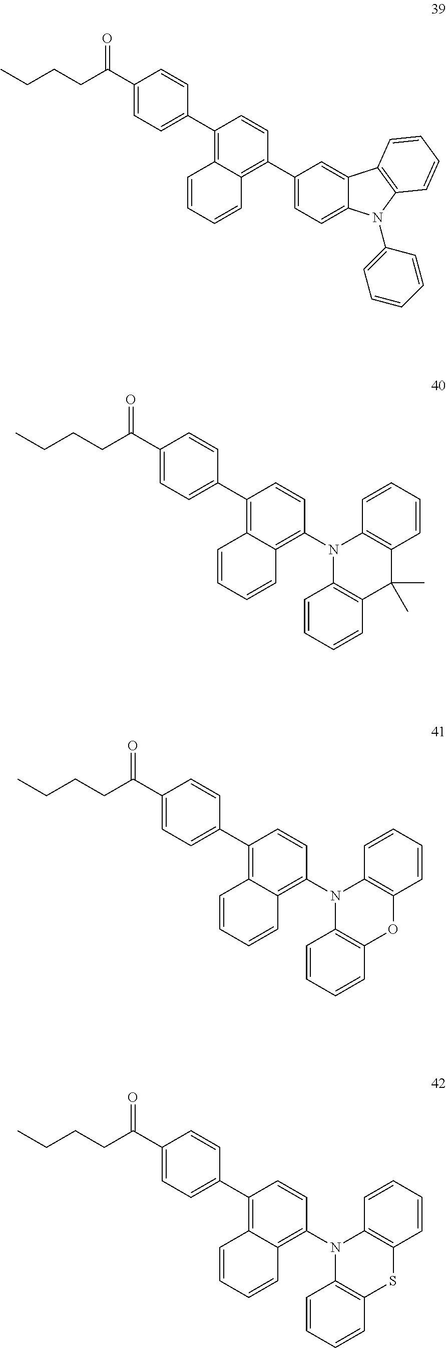

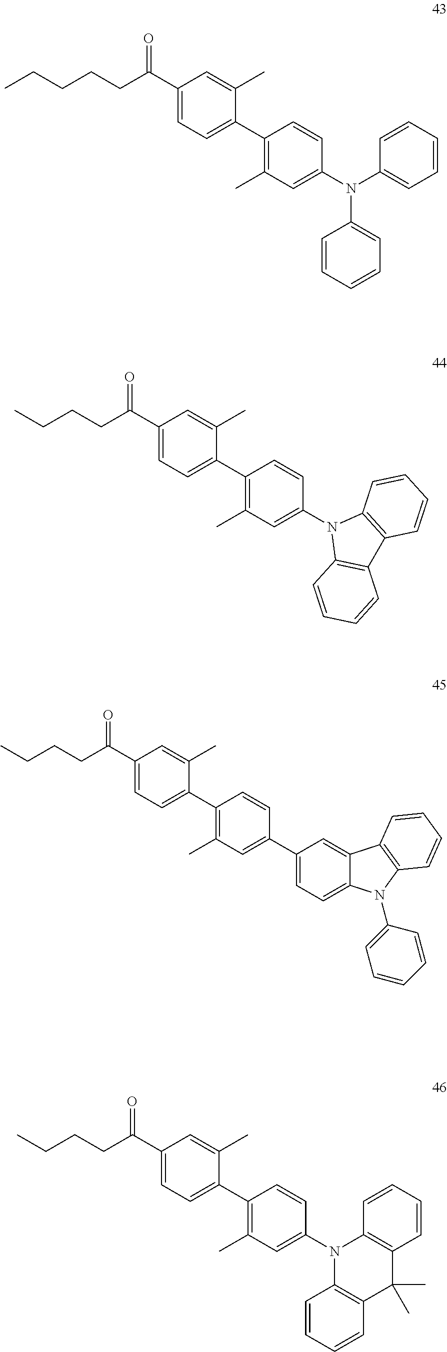 [SMILES: CCCCCC(=O)c1ccc(-c2ccc(N(c3ccccc3)c3ccccc3)cc2C)c(C)c1 ; CCCCC(=O)c1ccc(-c2ccc(-n3c4ccccc4c4ccccc43)cc2C)c(C)c1 ; CCCCC(=O)c1ccc(-c2ccc(-c3ccc4c(c3)c3ccccc3n4-c3ccccc3)cc2C)c(C)c1 ; CCCCC(=O)c1ccc(-c2ccc(N3c4ccccc4C(C)(C)c4ccccc43)cc2C)c(C)c1]

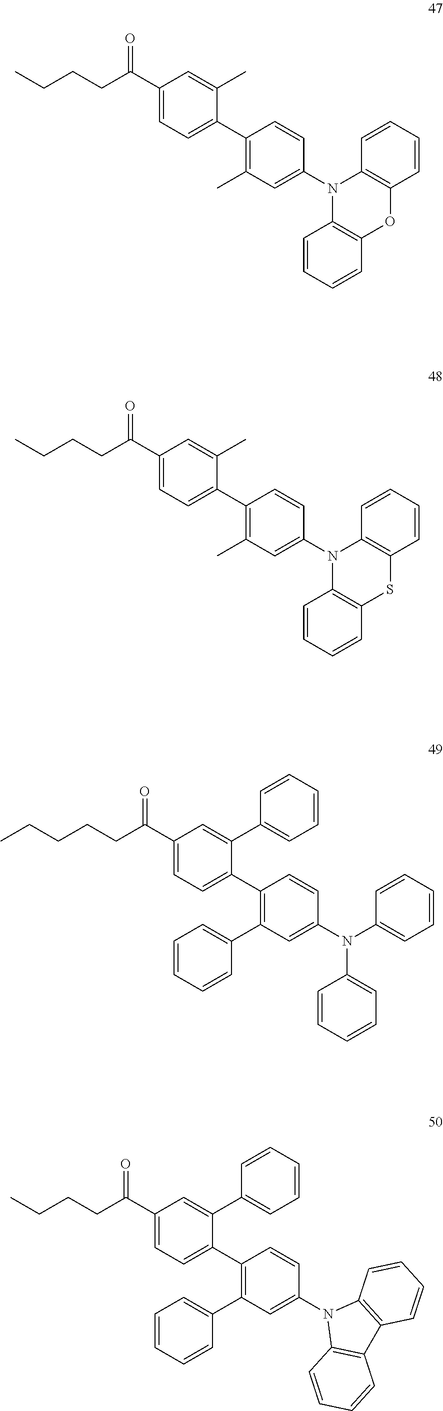 [SMILES: CCCCC(=O)c1ccc(-c2ccc(N3c4ccccc4Oc4ccccc43)cc2C)c(C)c1 ; CCCCC(=O)c1ccc(-c2ccc(N3c4ccccc4Sc4ccccc43)cc2C)c(C)c1 ; CCCCCC(=O)c1ccc(-c2ccc(N(c3ccccc3)c3ccccc3)cc2-c2ccccc2)c(-c2ccccc2)c1 ; CCCCC(=O)c1ccc(-c2ccc(-n3c4ccccc4c4ccccc43)cc2-c2ccccc2)c(-c2ccccc2)c1]

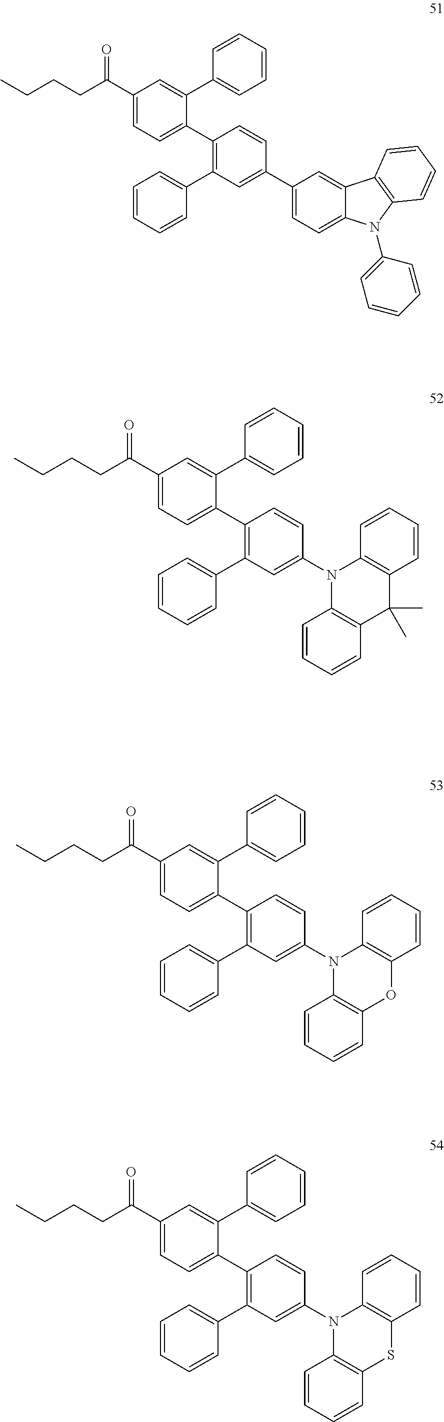

Preferably the following compound:

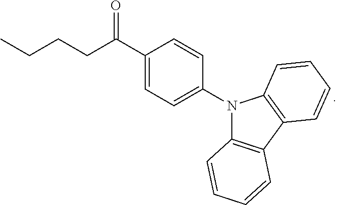 [SMILES: CCCCC(=O)c1ccc(-n2c3ccccc3c3ccccc32)cc1]

The one or more organic layers are one or more of a hole injection layer, a hole transport layer, a light-emitting layer, a hole blocking layer and an electron transport layer respectively.

The material having the formula (I) is used in the hole injection layer, the hole transport layer and/or the light-emitting layer.

When the material having the formula (I) is used as a hole injection material or a hole transport material, a single material may be used as a single layer or a plurality of materials may be combined to form a single layer.

When the material having the formula (I) is used as a light-emitting layer, the material can be used as a single light-emitting layer or doped with other materials as a light-emitting layer.

The organic layer can form a thin film by vacuum coating or spin-coating.

Since the compound having the formula (I) in the present invention has electron-withdrawing groups and electron-donating groups with high stability, stable chemical bonds exist between them, without strong conjugation. With these characteristics, the compound having the formula (I) has higher fluorescence quantum efficiency and excellent charge transport ability, and closer to the international standard of dark blue. When used in OLEDs, devices will have better light-emitting performance.

### BRIEF DESCRIPTION OF THE DRAWINGS

FIG. 1 is a structural diagram of the device in the invention; 10 represents a glass substrate, 20 represents an anode ITO, 30 represents a hole transport layer, 40 represents an electron/exciton blocking layer, 50 represents a light emitting layer, 60 represent an exciton blocking layer, 70 represents an electron transport layer, 80 represents an electron injection layer, and 90 represents a cathode.

FIG. 2 is a 1H-NMR diagram of compound 1.

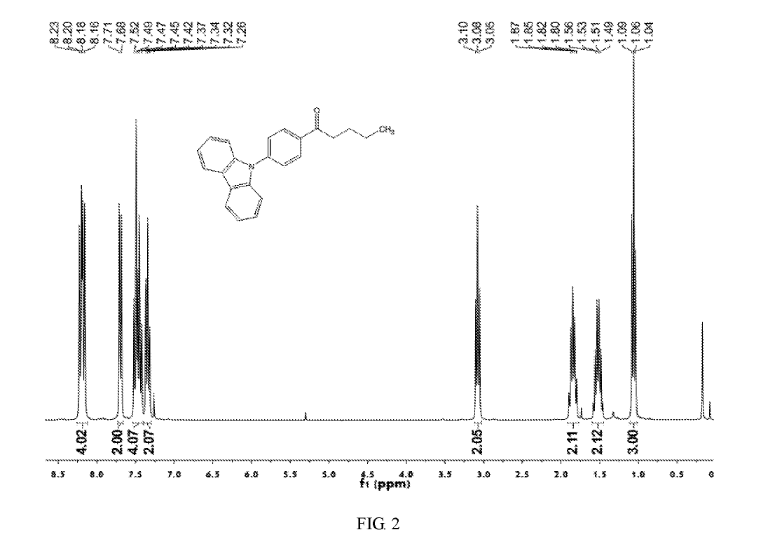

### DETAILED DESCRIPTIONS OF THE PREFERRED EMBODIMENTS

The present invention is further described in combination with embodiments, but it should not be construed as limiting the invention in any way.

### Embodiment 1: Synthesis of Compound 1

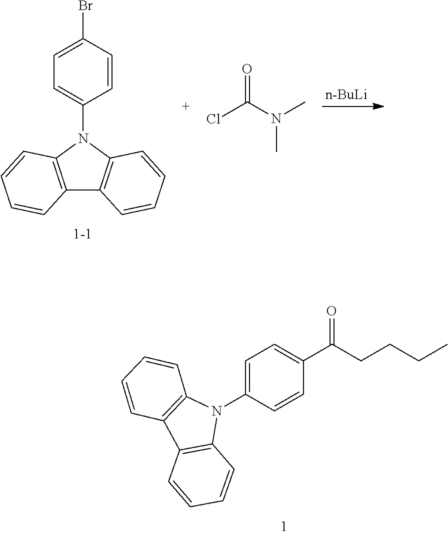

To a reaction vessel, add 2 g (0.018 mol) of dimethylcarbamoyl chloride and 20 ml of tetrahydrofuran solvent, then oxygen purging and nitrogen protection is performed for the device. Cool down the temperature of the reaction solution to −75˜65° C., slowly add 10 ml of 1.6M n-BuLi/THF solution dropwise, to control the temperature of the reaction solution at −75˜65° C. After dripping, continue to maintain this temperature to react 0.5-1 h. After the addition of 6 g of compound 1-1, control the temperature of the reaction solution at −75˜65° C. After dripping, continue to maintain this temperature to react 0.5-1 h, then transfer the reaction solution to room temperature and naturally heat 4-6 h, then stop the reaction. Add ethyl acetate/deionized water to extract and extract the aqueous layer with ethyl acetate. Combine the organic layer, dry with anhydrous magnesium sulfate, filter, concentrate the filtrate to get an off-white solid. Conduct chromatography for the resulting solids, to get 3 g of white solid powder.

### Embodiment 2: Application Example of the Compound in the Invention

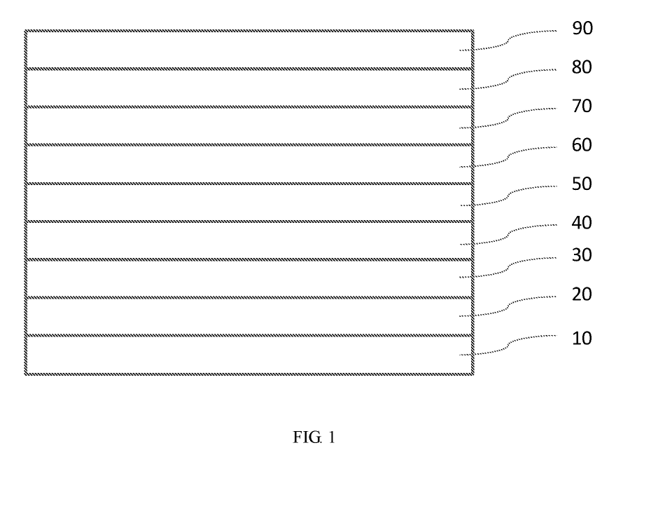

The device structure is shown in FIG. 1.

Device Preparation:

Firstly, the ITO transparent conductive glass substrate 10 (with anode 20 above) is washed with detergent solution and deionized water, acetone ultrasound, isopropanol vapor, and then treated with oxygen plasma for 5 minutes.

Then, perform vacuum evaporation of 35 nm NPB in ITO, which is used as the hole injection layer 30.

Then, perform vacuum evaporation of 5 nm of mCP as the electron/exciton blocking layer 40.

Then, perform vacuum evaporation of 20 nm of light-emitting layer 50, using mCP as a host material and compound 1 in the invention as a doped material, with a doping concentration of 3%.

Then, perform vacuum evaporation of 10 nm of mCP as the exciton blocking layer 60.

Then, perform vacuum evaporation of 30 nm of TPBi as the electron transport layer 70.

Finally, perform vacuum evaporation of 1 nm of LiF as the electron injection layer and vacuum evaporation of 100 nm of Al as the cathode 90.

The OLEDs prepared in the invention emit near ultraviolet light, with the emission wavelength of 416 nm and color coordinate (0.17, 0.09).

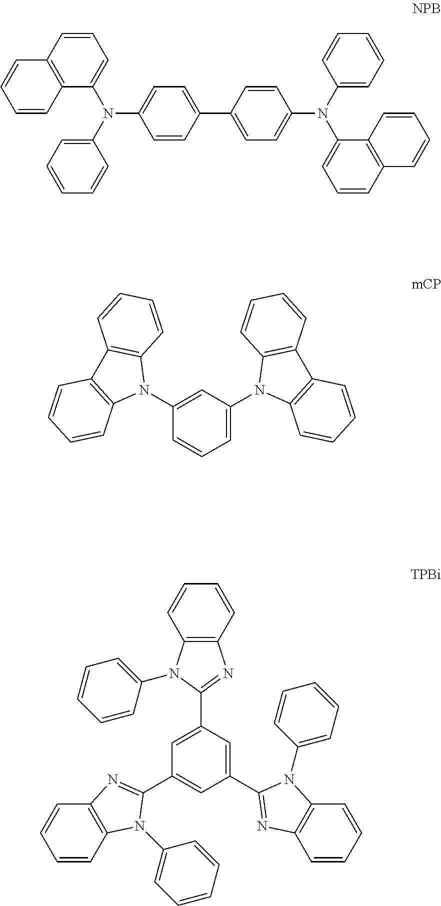 [SMILES: c1ccc(N(c2ccc(-c3ccc(N(c4ccccc4)c4cccc5ccccc45)cc3)cc2)c2cccc3ccccc23)cc1 ; c1cc(-n2c3ccccc3c3ccccc32)cc(-n2c3ccccc3c3ccccc32)c1 ; c1ccc(-n2c(-c3cc(-c4nc5ccccc5n4-c4ccccc4)cc(-c4nc5ccccc5n4-c4ccccc4)c3)nc3ccccc32)cc1]

### Comparison Example

In the Comparison Example, the compound Cz2BP reported in the literature Angew. Chem. Int. Ed. 2014, 53, 6402-6406 was used to replace the compound 1. The device structure is consistent with that in the document, and basically the same as the device in embodiment 6. The device adopts DPEPO except host material and exciton blocking material. In the comparison example, the emission wavelength of the OLEDs is 446 nm and the color coordinate is (0.16, 0.14).

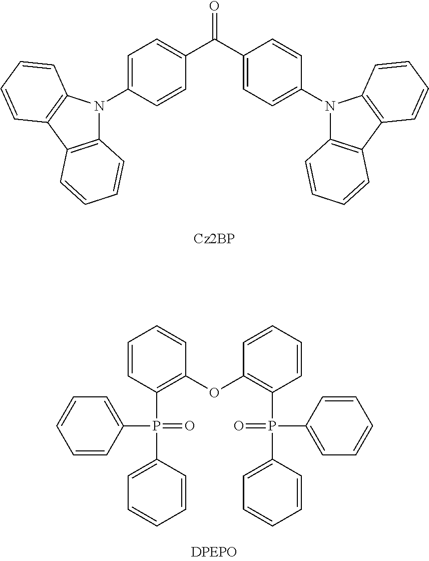 [SMILES: O=C(c1ccc(-n2c3ccccc3c3ccccc32)cc1)c1ccc(-n2c3ccccc3c3ccccc32)cc1 ; O=P(c1ccccc1)(c1ccccc1)c1ccccc1Oc1ccccc1P(=O)(c1ccccc1)c1ccccc1]

Therefore, the material in the invention has a color coordinate that is closer to the National Television Standards Committee (NTSC) standard for dark blue (0.14, 0.08), compared to the reported materials.

1. An organic light emitting device (OLED), comprising an anode, a cathode and one or more organic layers, and at least one of the organic layers comprising a material having the following chemical formula (I): 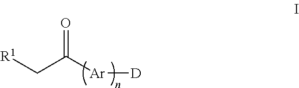
wherein;
Ar is unsubstituted or at least one R4-substituted benzene ring, naphthalene ring, anthracene ring, n=0-3;
D is an electron donating group containing a nitrogen atom and is one of the following groups: 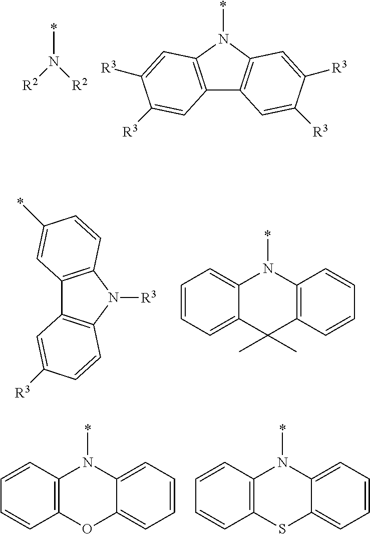
when D is —N(R²)₂, n is not zero;
R¹, R³, R⁴ are each independently selected from hydrogen H, deuterium D, fluorine F, chlorine Cl, bromine Br, iodine I, hydroxy OH, cyano CN, amino NH2, nitro NO₂, cyclic or non-cyclic alkyl group containing 1 to 20 carbon atoms, cyclic or non-cyclic alkoxy containing 1 to 20 carbon atoms, C6-C40 R⁵-substituted or unsubstituted aryl containing one or more substituents, C6-C40 R⁵-substituted or unsubstituted aryl alkyl containing one or more substituents, C5-C40 R⁵-substituted or unsubstituted heteroaryl groups containing one or more heteroatoms; two or more of the R²-R⁴ groups may be bonded to each other to form a ring;
R² is independently selected from C6-C40 R⁵ substituted or unsubstituted aryl containing one or more substituents, C6-C40 R⁵ substituted or unsubstituted aryl alkyl containing one or more substituents, C5-C40 R⁵-substituted or unsubstituted heteroaryl groups containing one or more heteroatoms;
R⁵ is selected from fluorine F, chlorine Cl, bromide Br, iodine I, hydroxy OH, cyano CN, amino NH₂, nitro NO₂, cyclic or non-cyclic alkyl containing 1 to 20 carbon atoms; and
the heteroatoms are B, O, S, Se, N, P.

2. The OLED according to claim 1, wherein:
R¹, R³, R⁴ are each independently selected from hydrogen H, deuterium D, fluorine F, chlorine Cl, bromine Br, iodine I, hydroxy OH, cyano CN, amino NH2, nitro NO₂, alkyl group containing 1 to 4 carbon atoms, alkoxy containing 1 to 4 carbon atoms, C6-C25 R⁵-substituted or unsubstituted aryl containing one or more substituents, C6-C25 R⁵-substituted or unsubstituted aryl alkyl containing one or more substituents, C5-C25 R⁵-substituted or unsubstituted heteroaryl groups containing one or more heteroatoms; R² is independently selected from C6-C25 R⁵ substituted or unsubstituted aryl containing one or more substituents, C6-C25 R⁵ substituted or unsubstituted aryl alkyl containing one or more substituents, C5-C25 R⁵-substituted or unsubstituted heteroaryl groups containing one or more heteroatoms; two or more of the R¹-R⁴ groups may be bonded to each other to form a ring;
R⁵ is selected from hydrogen H, fluorine F, chlorine Cl, bromide Br, iodine I, hydroxy OH, cyano CN, amino NH₂, nitro NO₂, alkyl containing 1 to 4 carbon atoms; and
the heteroatoms are O, S, N; and
Ar is unsubstituted or one R⁴ substituted benzene ring, naphthalene ring, anthracene ring.

3. The OLED according to claim 2, wherein:
R¹, R³, R⁴ are each independently selected from hydrogen H, fluorine F, chlorine Cl, bromine Br, alkyl group containing 1 to 4 carbon atoms, C6-C25 R⁵-substituted or unsubstituted aryl containing one or more substituents, C6-C25 R⁵-substituted or unsubstituted aryl alkyl containing one or more substituents, C5-C25 R⁵-substituted or unsubstituted heteroaryl groups containing one or more heteroatoms; R² is independently selected from C6-C25 R⁵ substituted or unsubstituted aryl containing one or more substituents, C5-C25 R⁵-substituted or unsubstituted heteroaryl groups containing one or more heteroatoms; two or more of the R¹-R⁴ groups may be bonded to each other to form a ring;
R⁵ is selected from hydrogen H, fluorine F, chlorine Cl, bromide Br, alkyl containing 1 to 4 carbon atoms; and
the heteroatoms are O, S, N.

4. The OLED according to claim 3, wherein:
R¹ is selected from alkyl containing 1 to 4 carbon atoms, R5 substituted or unsubstituted phenyl, naphthyl, anthryl containing one or more substituents;
R² is independently R5 substituted or unsubstituted phenyl or naphthyl or anthryl containing one or more substituents;
R³ is selected from hydrogen H, fluorine F, chlorine Cl, bromine Br, iodine I, alkyl having 1 to 4 carbon atoms, R5 substituted or unsubstituted phenyl or naphthyl or anthryl containing one or more substituents;
R⁴ is selected from hydrogen H, fluorine F, chlorine Cl, bromine Br, iodine I, alkyl, phenyl, naphthyl containing 1 to 4 carbon atoms; and
R⁵ is selected from hydrogen H, alkyl containing 1 to 4 carbon atoms.

5. The OLED according to claim 4, wherein:
R¹ is selected from alkyl containing 1 to 4 carbon atoms;
R² is independently phenyl or naphthyl or anthryl;
R³ is selected from hydrogen H, phenyl or naphthyl or anthryl containing 1 to 4 carbon atoms; and
R⁴ is selected from hydrogen H, phenyl or naphthyl or anthryl containing 1 to 4 carbon atoms.

6. The OLED according to claim 5, wherein the material having the chemical formula (I) is selected from the group consisting of the following compounds:

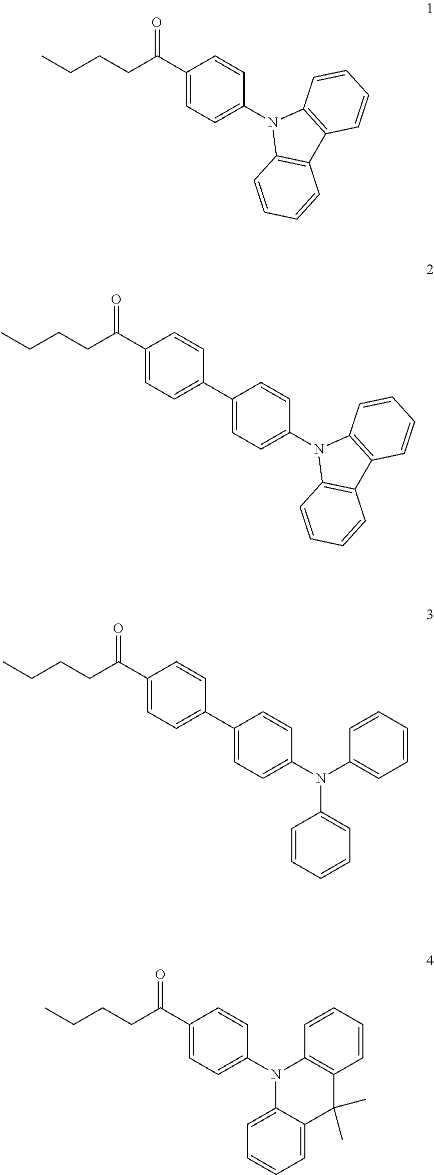 [SMILES: CCCCC(=O)c1ccc(-n2c3ccccc3c3ccccc32)cc1 ; CCCCC(=O)c1ccc(-c2ccc(-n3c4ccccc4c4ccccc43)cc2)cc1 ; CCCCC(=O)c1ccc(-c2ccc(N(c3ccccc3)c3ccccc3)cc2)cc1 ; CCCCC(=O)c1ccc(N2c3ccccc3C(C)(C)c3ccccc32)cc1]

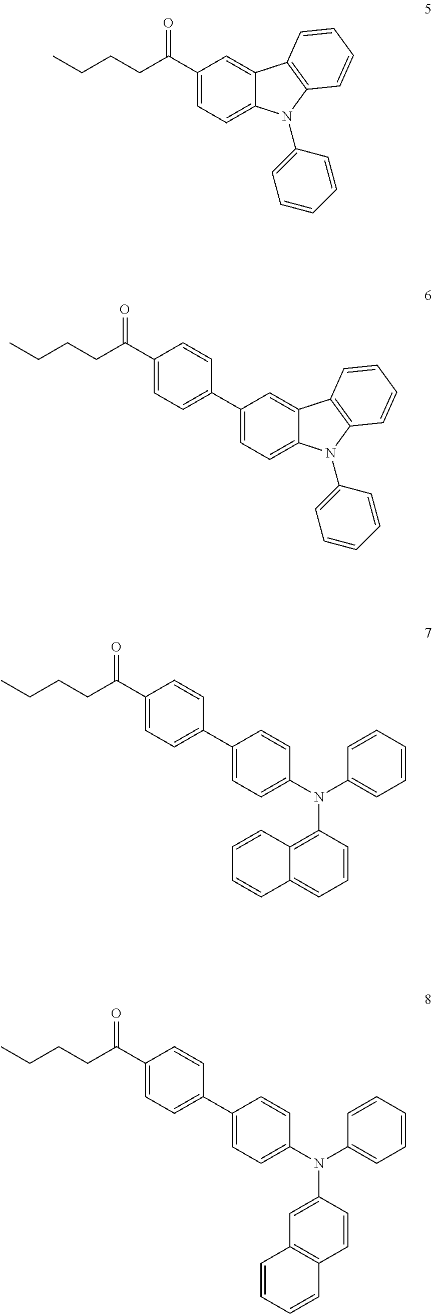 [SMILES: CCCCC(=O)c1ccc2c(c1)c1ccccc1n2-c1ccccc1 ; CCCCC(=O)c1ccc(-c2ccc3c(c2)c2ccccc2n3-c2ccccc2)cc1 ; CCCCC(=O)c1ccc(-c2ccc(N(c3ccccc3)c3cccc4ccccc34)cc2)cc1 ; CCCCC(=O)c1ccc(-c2ccc(N(c3ccccc3)c3ccc4ccccc4c3)cc2)cc1]

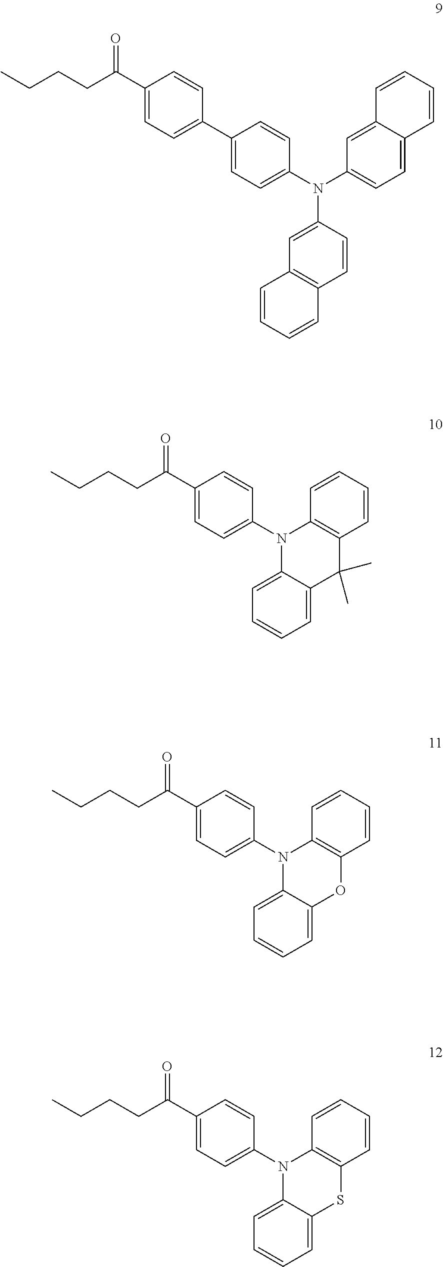

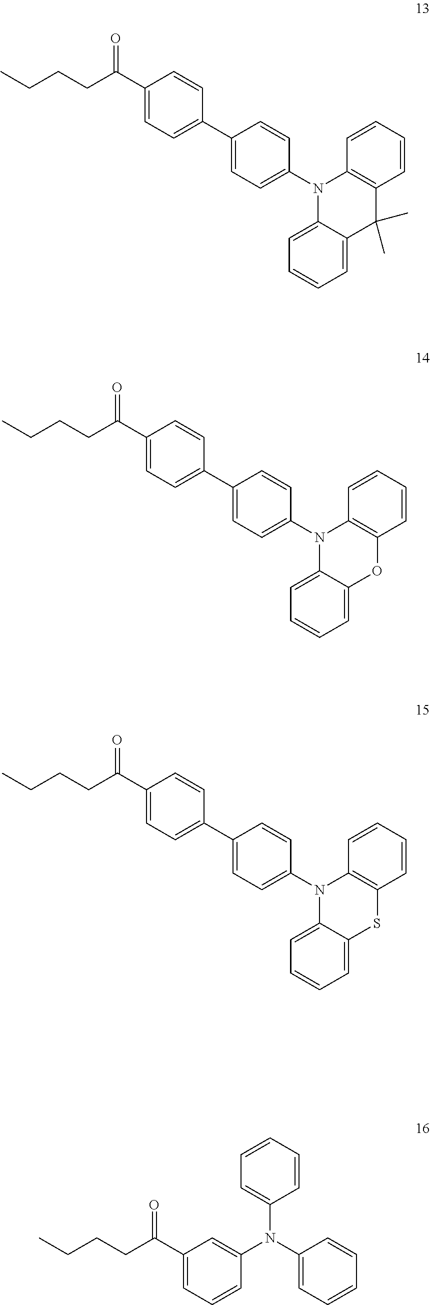

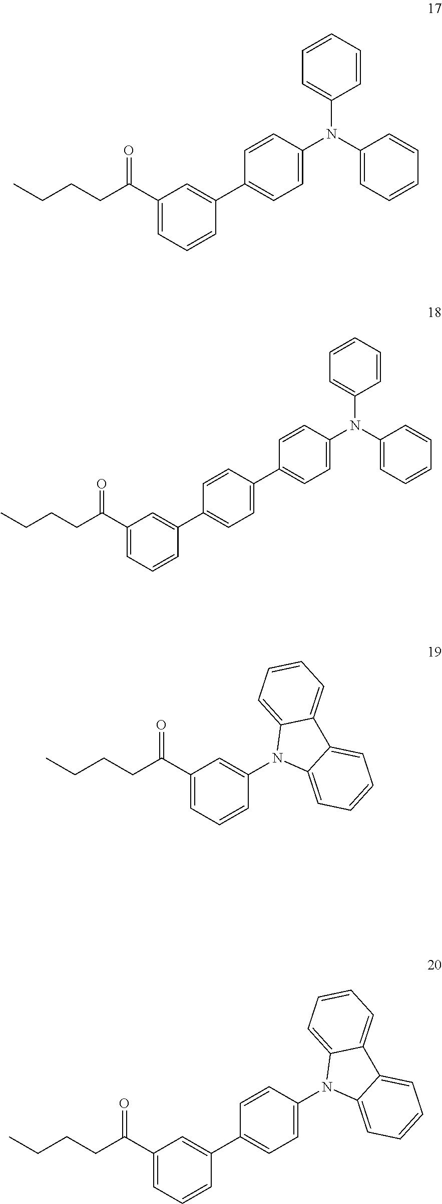 [SMILES: CCCCC(=O)c1cccc(-c2ccc(N(c3ccccc3)c3ccccc3)cc2)c1 ; CCCCC(=O)c1cccc(-c2ccc(-c3ccc(N(c4ccccc4)c4ccccc4)cc3)cc2)c1 ; CCCCC(=O)c1cccc(-n2c3ccccc3c3ccccc32)c1 ; CCCCC(=O)c1cccc(-c2ccc(-n3c4ccccc4c4ccccc43)cc2)c1]

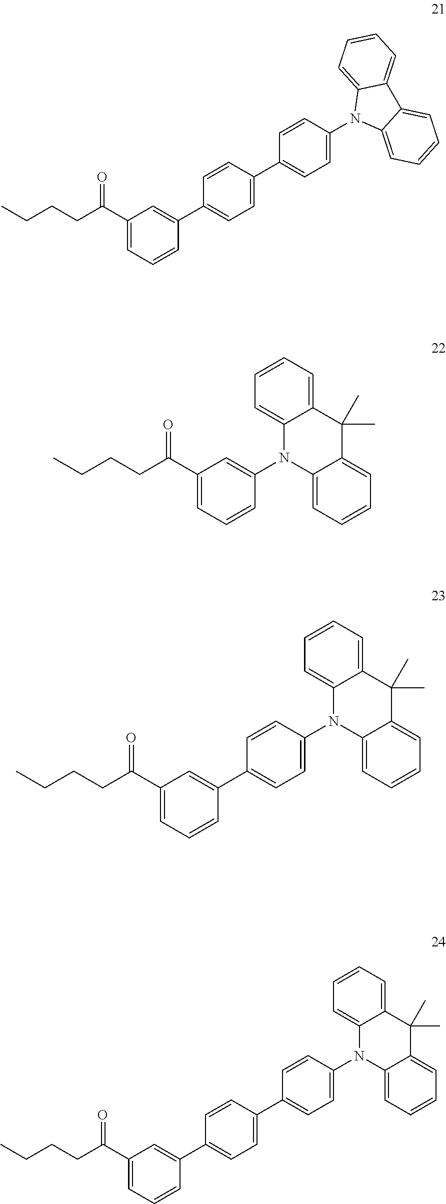

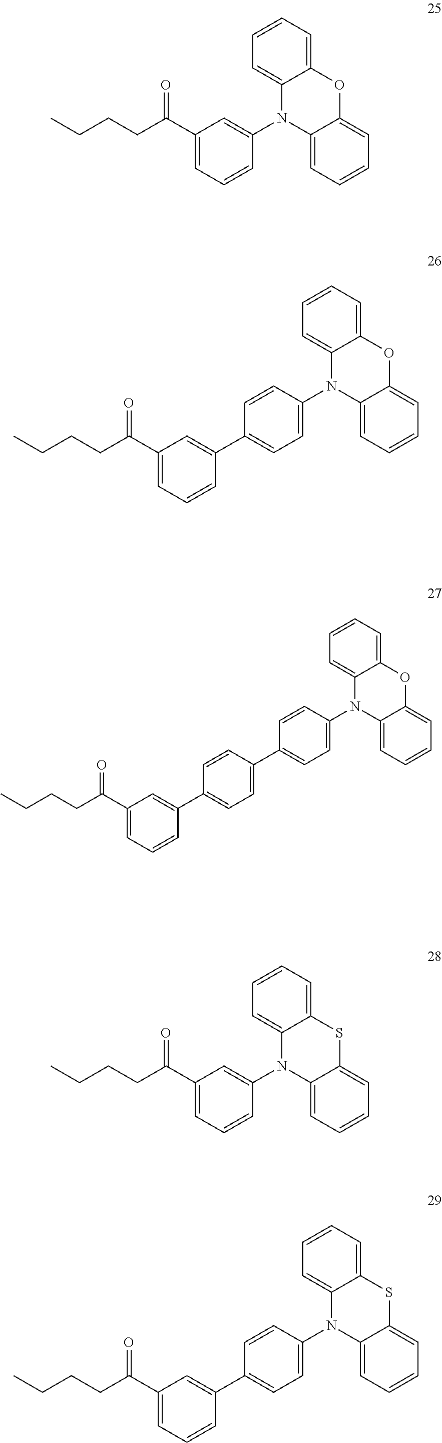 [SMILES: CCCCC(=O)c1cccc(N2c3ccccc3Oc3ccccc32)c1 ; CCCCC(=O)c1cccc(-c2ccc(N3c4ccccc4Oc4ccccc43)cc2)c1 ; CCCCC(=O)c1cccc(-c2ccc(-c3ccc(N4c5ccccc5Oc5ccccc54)cc3)cc2)c1 ; CCCCC(=O)c1cccc(N2c3ccccc3Sc3ccccc32)c1 ; CCCCC(=O)c1cccc(-c2ccc(N3c4ccccc4Sc4ccccc43)cc2)c1]

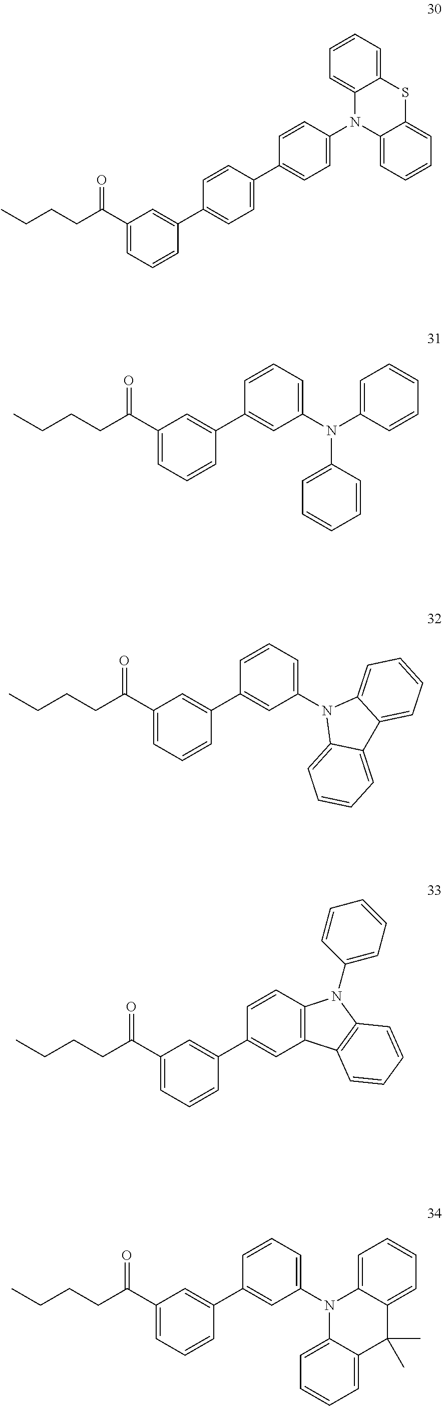 [SMILES: CCCCC(=O)c1cccc(-c2ccc(-c3ccc(N4c5ccccc5Sc5ccccc54)cc3)cc2)c1 ; CCCCC(=O)c1cccc(-c2cccc(N(c3ccccc3)c3ccccc3)c2)c1 ; CCCCC(=O)c1cccc(-c2cccc(-n3c4ccccc4c4ccccc43)c2)c1 ; CCCCC(=O)c1cccc(-c2ccc3c(c2)c2ccccc2n3-c2ccccc2)c1 ; CCCCC(=O)c1cccc(-c2cccc(N3c4ccccc4C(C)(C)c4ccccc43)c2)c1]

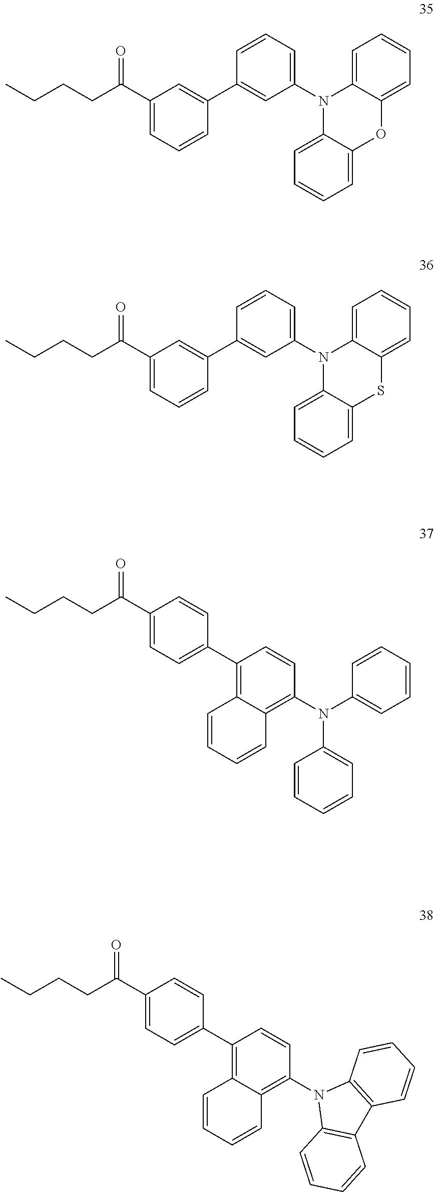 [SMILES: CCCCC(=O)c1cccc(-c2cccc(N3c4ccccc4Oc4ccccc43)c2)c1 ; CCCCC(=O)c1cccc(-c2cccc(N3c4ccccc4Sc4ccccc43)c2)c1 ; CCCCC(=O)c1ccc(-c2ccc(N(c3ccccc3)c3ccccc3)c3ccccc23)cc1 ; CCCCC(=O)c1ccc(-c2ccc(-n3c4ccccc4c4ccccc43)c3ccccc23)cc1]

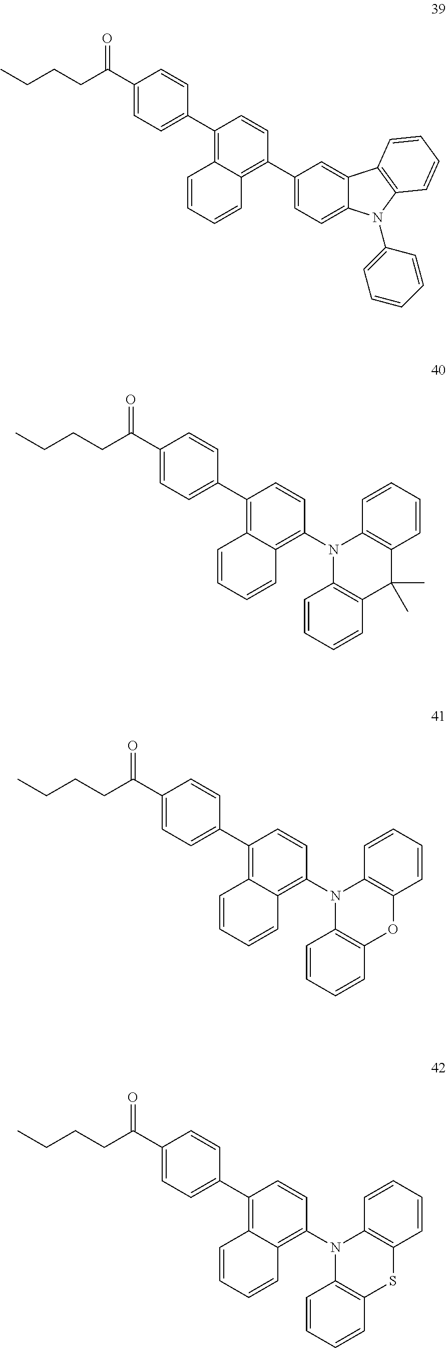

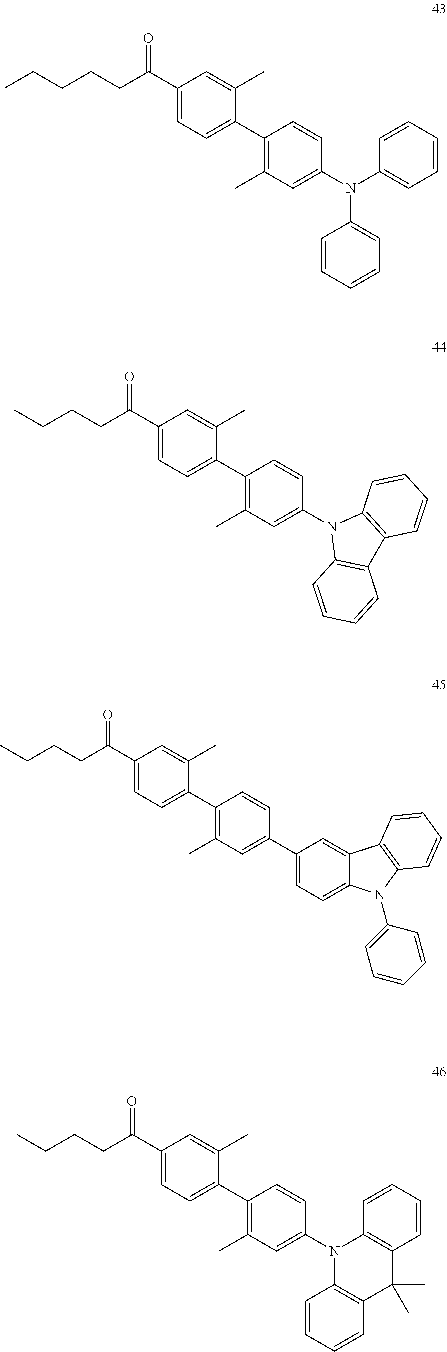 [SMILES: CCCCCC(=O)c1ccc(-c2ccc(N(c3ccccc3)c3ccccc3)cc2C)c(C)c1 ; CCCCC(=O)c1ccc(-c2ccc(-n3c4ccccc4c4ccccc43)cc2C)c(C)c1 ; CCCCC(=O)c1ccc(-c2ccc(-c3ccc4c(c3)c3ccccc3n4-c3ccccc3)cc2C)c(C)c1 ; CCCCC(=O)c1ccc(-c2ccc(N3c4ccccc4C(C)(C)c4ccccc43)cc2C)c(C)c1]

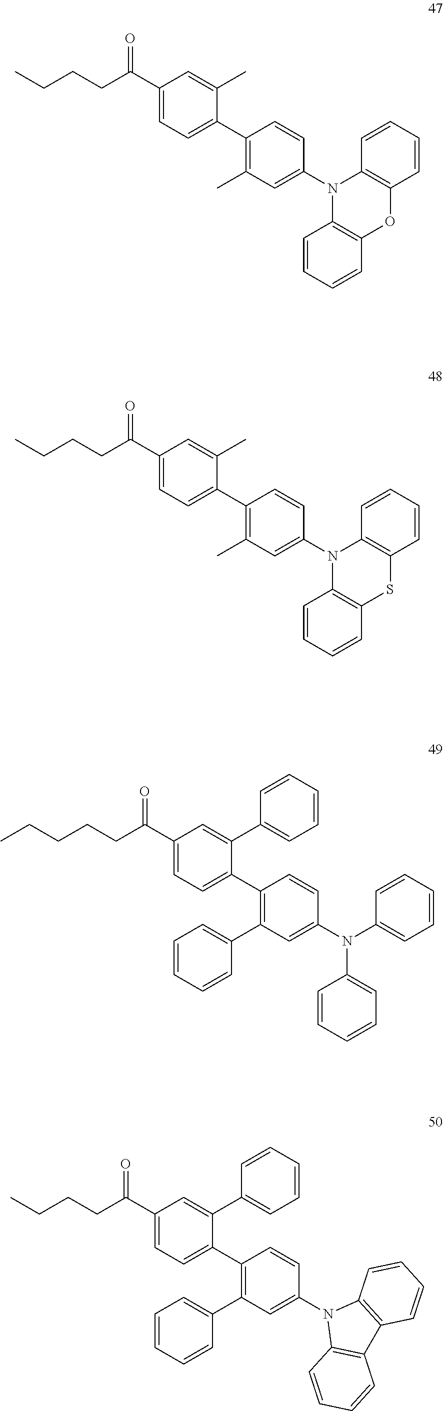 [SMILES: CCCCC(=O)c1ccc(-c2ccc(N3c4ccccc4Oc4ccccc43)cc2C)c(C)c1 ; CCCCC(=O)c1ccc(-c2ccc(N3c4ccccc4Sc4ccccc43)cc2C)c(C)c1 ; CCCCCC(=O)c1ccc(-c2ccc(N(c3ccccc3)c3ccccc3)cc2-c2ccccc2)c(-c2ccccc2)c1 ; CCCCC(=O)c1ccc(-c2ccc(-n3c4ccccc4c4ccccc43)cc2-c2ccccc2)c(-c2ccccc2)c1]

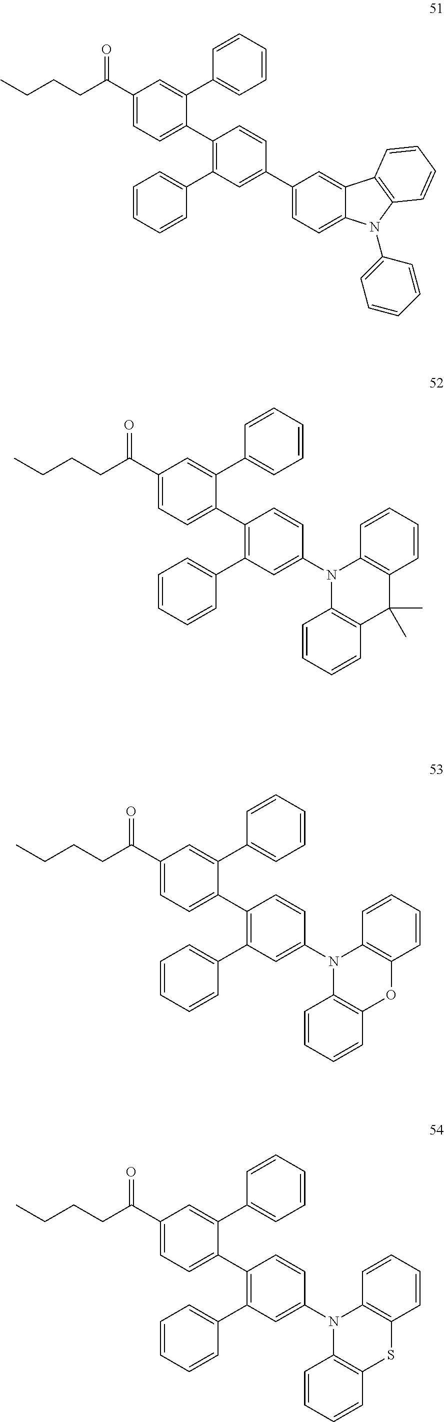

7. The OLED according to claim 2, wherein the material having the chemical formula (I) is the following compound:

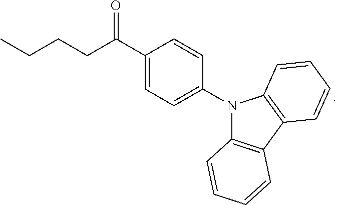 [SMILES: CCCCC(=O)c1ccc(-n2c3ccccc3c3ccccc32)cc1]

8. The OLED according to claim 1, wherein the one or more organic layers are one or more of a hole injection layer, a hole transport layer, a light-emitting layer, a hole blocking layer and an electron transport layer.

9. The OLED according to claim 8, wherein the material having the formula (I) can be used as a single light-emitting layer or doped with other materials as a light-emitting layer when used as a light-emitting layer.

10. The OLED according to claim 2, wherein the one or more organic layers are one or more of a hole injection layer, a hole transport layer, a light-emitting layer, a hole blocking layer and an electron transport layer.

11. The OLED according to claim 10, wherein the material having the formula (I) can be used as a single light-emitting layer or doped with other materials as a light-emitting layer when used as a light-emitting layer.

12. The OLED according to claim 3, wherein the one or more organic layers are one or more of a hole injection layer, a hole transport layer, a light-emitting layer, a hole blocking layer and an electron transport layer.

13. The OLED according to claim 12, wherein the material having the formula (I) can be used as a single light-emitting layer or doped with other materials as a light-emitting layer when used as a light-emitting layer.

14. The OLED according to claim 4, wherein the one or more organic layers are one or more of a hole injection layer, a hole transport layer, a light-emitting layer, a hole blocking layer and an electron transport layer.

15. The OLED according to claim 14, wherein the material having the formula (I) can be used as a single light-emitting layer or doped with other materials as a light-emitting layer when used as a light-emitting layer.

16. The OLED according to claim 5, wherein the one or more organic layers are one or more of a hole injection layer, a hole transport layer, a light-emitting layer, a hole blocking layer and an electron transport layer.

17. The OLED according to claim 16, wherein the material having the formula (I) can be used as a single light-emitting layer or doped with other materials as a light-emitting layer when used as a light-emitting layer.

18. The OLED according to claim 6, wherein the one or more organic layers are one or more of a hole injection layer, a hole transport layer, a light-emitting layer, a hole blocking layer and an electron transport layer.

19. The OLED according to claim 18, wherein the material having the formula (I) can be used as a single light-emitting layer or doped with other materials as a light-emitting layer when used as a light-emitting layer.

20. The OLED according to claim 7, wherein the one or more organic layers are one or more of a hole injection layer, a hole transport layer, a light-emitting layer, a hole blocking layer and an electron transport layer.
